(19) United States
(12) Patent Application Publication
(10) Pub. No.: US 2024/0050403 A1
(43) Pub. Date: Feb. 15, 2024

(54) THERAPY OF POST-OPERATIVE EMESIS WITH AMISULPRIDE

(71) Applicant: Acacia Pharma Limited, Cambridge (GB)

(72) Inventors: Julian Clive GILBERT, Cambridge (GB); Robert William GRISTWOOD, Cambridge (GB); Gabriel FOX, Cambridge (GB)

(21) Appl. No.: 18/364,796

(22) Filed: Aug. 3, 2023

Related U.S. Application Data
(63) Continuation of application No. 16/346,563, filed on May 1, 2019, now abandoned.

(30) Foreign Application Priority Data
Nov. 1, 2016 (GB) 1618425.1

(51) Int. Cl. A61K 31/40; A61P 1/08; A61K 31/496; A61K 31/4178; A61K 31/573; A61K 31/439; A61K 31/454; A61K 31/473; A61K 31/5377; A61K 45/06

(52) U.S. Cl. CPC A61K 31/40; A61P 1/08; A61K 31/496; A61K 31/4178; A61K 31/573; A61K 31/439; A61K 31/454; A61K 31/473; A61K 31/5377; A61K 45/06

(57) ABSTRACT

Amisulpride is useful in the therapy (particularly the prevention) of postoperative emesis in a patient, particularly wherein the patient is undergoing a surgical procedure where postoperative emesis would be potentially dangerous to the patient.

### CROSS REFERENCE TO RELATED APPLICATIONS

This application is a Continuation of U.S. patent application Ser. No. 16/346,563, filed May 1, 2019, which is a U.S. National Stage of PCT/GB2017/053288, filed Nov. 1, 2017, which claims priority to GB 1618425.1, filed Nov. 1, 2016, the entirety of which are incorporated by reference herein.

### FIELD OF THE INVENTION

This invention relates to the therapy of postoperative emesis.

### BACKGROUND OF THE INVENTION

Postoperative emesis is a subset of postoperative nausea and vomiting (PONV). PONV is a condition that occurs in approximately 30% of all surgical patients and 70% of high-risk patients. Risk factors for PONV include: type of surgery, sex, smoking history, prior history of PONV or motion sickness, length of surgery, use of volatile anaesthetics and opioid analgesic usage. Typically, women are more prone than men to PONV, as are non-smokers and those who have previously experienced PONV or motion sickness.

PONV is a significant issue for patients and healthcare providers. It is often rated above postoperative pain as a complication most feared by patients and thus contributes significantly to anxiety and patient distress. PONV can delay discharge of the patient from hospital or result in readmission after in-patient procedures and can require admission for ambulatory patients. This has a significant economic and social impact. With increasing rates of hospital-acquired resistant infections, it may also translate into an impact on clinical outcomes.

Numerous mechanisms have been implicated in PONV, most notably release of serotonin from the gut wall and activation of the chemoreceptor trigger zone in the brain. Consequently, several different receptors seem to be involved in PONV and represent effective targets for drug therapies. Among the most important are the serotoninergic 5HT₃ and the dopaminergic D2 and possibly D3 receptors.

Despite routine use of prophylactic anti-emetics in moderate and high-risk patients, PONV still occurs in about 30% of cases, even in patients receiving the newest agents and there remains a significant need for additional agents, especially with different mechanisms of action.

The use of amisulpride as an anti-emetic is described in WO2011/110854, published on 15 Sep. 2011, which claims priority from British Patent Specification, GB 1004020.2, filed on 11 Mar. 2010. Both of these documents are incorporated into this present specification in their entirety.

In a multi-centre, double-blind, randomised, placebo-controlled Phase II clinical trial in adult surgical patients (conducted by the applicant), administration of amisulpride at 5 mg was associated with a PONV incidence of 40%, compared to 69% with placebo (p<0.01). There was no difference in the nature, incidence or severity of adverse events, or of laboratory or ECG abnormalities, between amisulpride and placebo.

In two multi-centre, double-blind, randomised, placebo-controlled Phase III clinical trials involving 626 evaluable, adult surgical patients (again conducted by the applicant), administration of amisulpride at 5 mg was associated with a PONV incidence of 48%, compared to 59% with placebo (p<0.01). There was no significant difference between the safety profiles of amisulpride and placebo, except that transient increases in serum prolactin levels were more common with amisulpride.

Postoperative emesis is particularly problematic in particular patient groups. This is because it increases the risk of pulmonary aspiration and has been associated with suture dehiscence, esophageal rupture, subcutaneous emphysema, and bilateral pneumothoraxes. Postoperative emesis can also lead to venous hypertension, increased intracranial pressure (ICP), and hematomas. Therefore, there are particular surgical procedures where postoperative emesis would pose great potential risk to patients.

### SUMMARY OF THE INVENTION

The present invention is based on the results of a Phase III study of amisulpride as prophylaxis against PONV in high-risk patients, conducted by the applicant. As expected, amisulpride was found to be efficacious in the therapy of PONV, but upon detailed analysis of the data (in particular the secondary efficacy analyses), it was surprisingly found that the relative risk reduction (RRR) of the incidence of emesis was much higher than expected (when compared to RRR of the overall risk of PONV, for example). Therefore, amisulpride is particularly efficacious in the prevention of postoperative emesis.

There is a group of patients for which postoperative vomiting would be particularly problematic, and in some embodiments, the present invention is the realisation that the use of amisulpride in this sub-population of patients would be particularly beneficial. In some instances, amisulpride is particularly efficacious in the therapy of post-operative emesis where patient has at least three risk factors for post-operative emesis, wherein the risk factors are selected from a past history of postoperative nausea and vomiting and/or motion sickness; habitual non-smoking status; being a female; or expected use of post-operative opioid analgesia.

According to a first aspect, the present invention is amisulpride for use in the therapy of postoperative emesis in a patient. The patient may be selected from the group of patients undergoing a surgical procedure where postoperative emesis would be potentially dangerous to the patient.

According to a second aspect, a method for treating or preventing postoperative emesis in a patient, wherein the patient is undergoing a surgical procedure, comprises administering an effective amount of amisulpride to the patient and optionally pre-selecting a patient for the treatment or prevention, from a group of patients undergoing a surgical procedure where post-operative emesis would be potentially dangerous to the patient.

### DESCRIPTION OF THE INVENTION

Amisulpride has a single chiral centre and two enantiomers exist, i.e. (S−)-amisulpride and (R+)-amisulpride. It may be preferred to use the racemate or (S−)-amisulpride, which is substantially free of the (R+)-enantiomer. It has been reported that almost all of the therapeutic activity is to be found in the (S−)-enantiomer, and therefore use of this enantiomer means that it may be possible to reduce the dose by 50% (e.g., 50%, 60%, 70%, 80%, or 90%, or 50%-60%, 60%-70%, 70%-80%, or 80-90%) compared to the racemate.

A racemic mixture or racemate of amisulpride means that the amisulpride comprises both the (S−)-amisulpride and the (R+)-enantiomer. For example, the racemic mixture may comprise from 40% to 60% of (S−)-amisulpride and 60% to 40% of the (R+)-enantiomer. In some embodiments, the racemic mixture may comprise about 50% of (S−)-amisulpride and about 50% of the (R+)-enantiomer.

(S−)-amisulpride that is substantially free of the (R+)-enantiomer comprises less than 10%, less than 5%, less than 4%, less than 3%, less than 2%, or less than 1% of (R+)-enantiomer. For example, (S−)-amisulpride that is substantially free of the (R+)-enantiomer comprises less than 2% or less than 1% of (R+)-enantiomer.

As used herein, the term postoperative emesis means the occurrence of one or more emetic episodes (vomiting and/or retching). Retching involves the same physiological mechanisms as vomiting, but occurs against a closed glottis.

As used herein, the term “about” or “approximately”, when used together with a numeric value (e.g. 5, 10%, ⅓), refers to a range of numeric values that can be less or more than the number. For example, “about 5” refers to a range of numeric values that are 10%, 5%, 2%, or 1% less or more that 5, e.g. a range of 4.5 to 5.5, or 4.75 to 5.25, or 4.9 to 5.1, or 4.95 to 5.05. In some instances, “about 5” refers to a range of numeric values that are 2% or 1% less or more that 5, e.g. a range of 4.9 to 5.1 or 4.95 to 5.05.

Preferably, an effective amount (i.e. the dose) of amisulpride comprises 1 to 40 mg amisulpride, more preferably 1 to 20 mg or 2.5 to 20 mg, more preferably 5 to 10 mg and most preferably about 5 mg amisulpride. An effective amount of amisulpride may also comprise 2.5 to 5 mg, 2.5 to 10 mg, 2.5 to 40 mg, 5 to 20 mg, 5 to 40 mg, 1 to 5 mg or 1 to 10 mg amisulpride. Preferably, the amisulpride is in the form of a racemic mixture.

Preferably, an effective amount (i.e. the dose) of amisulpride comprises 1 to 20 mg amisulpride, more preferably 1 to 10 mg, more preferably 2.5 to 5 mg and most preferably about 2.5 mg amisulpride. An effective amount of amisulpride may also comprise 1 to 2.5 mg, 1 to 5 mg, 1 to 20 mg, 2.5 to 10 mg or 2.5 to 20 mg amisulpride. Preferably, the amisulpride is in the form of (S−)-amisulpride, and substantially free of the (R+)-enantiomer.

Preferably, amisulpride is administered as a single daily dose. More preferably, it is administered as a single dose.

It may be advantageous to administer amisulpride in combination with other classes of drugs which can add additional benefits of efficacy. Preferably, the other classes of drugs are different anti-emetic agents (i.e. an anti-emetic that is not amisulpride). More preferably, the different anti-emetic agent is not a D2 antagonist. These include, but are not limited to, steroids, most preferably dexamethasone, 5HT₃ antagonists including but not limited to ondansetron, granisetron and palonosetron, and NK₁ antagonists such as aprepitant, netupitant or rolapitant. Preferably, the other anti-emetic agent is ondansetron, granisetron or dexamethasone. Other classes of drugs may be administered via any appropriate routes of administration (e.g., via the route of administration which is typical for that drug, such as oral, intravenous or intramuscular). In some instances, other classes of drugs may be administered within 6 hours from the end of the surgery. In other instances, other classes of drugs may be administered after 6 hours from the end of the surgery.

Typical doses of the different anti-emetic agents listed above are known to a person skilled in the art. For example, ondansetron is typically in a dose of from 2 to 20 mg, or 2 to 15 mg, or about 10 mg or about 4 mg. For granisetron, the dose is typically 1-3 mg e.g. 1 mg. For dexamethasone, a typical dose is from 4-20 mg e.g. 4 mg.

Amisulpride for use according to the present invention may be packaged for sale together with accompanying instructions for use. The instructions for use (drug label) preferably specify in the list of indications that the drug can be used in a patient undergoing a surgical procedure where post-operative emesis would be potentially dangerous to the patient. It may specify the surgical procedures defined herein (for example, in the claims).

Amisulpride for use in the present invention is preferably formulated as an intravenous formulation (and intended for intravenous administration). The amisulpride may be in the form of a salt, hydrate or solvate. Salts include pharmaceutically acceptable salts, for example acid addition salts derived from inorganic or organic acids, such as hydrochlorides, hydrobromides, p-toluenesulphonates, phosphates, sulphates, perchlorates, acetates, trifluoroacetates, propionates, citrates, malonates, succinates, lactates, oxalates, tartrates and benzoates.

Salts may also be formed with bases. Such salts include salts derived from inorganic or organic bases, for example, alkali metal salts such as sodium and potassium salts and alkali earth metal salts such as magnesium and calcium salts, and organic amine salts, such as morpholine, piperidine, dimethylamine and diethylamine salts.

An intravenous formulation of amisulpride for use in the invention may be in the form of a sterile injectable aqueous or non-aqueous (e.g. oleaginous) solution or suspension. The sterile injectable preparation may also be in a sterile injectable solution or suspension in a non-toxic parenterally-acceptable diluent or solvent, for example, a solution in 1,3-butanediol. Among the acceptable vehicles and solvents that may be employed are water, phosphate buffer solution, Ringer's solution and isotonic sodium chloride solution. In addition, sterile, fixed oils may be used as a solvent or suspending medium. For this purpose, any bland fixed oil may be employed, including synthetic mono- or diglycerides. In addition, fatty acids such as oleic acid may be used in the preparation of the intravenous formulation of the invention. Suspensions may be formulated according to the known art using those suitable dispersing or wetting agents and suspending agents.

Aqueous suspensions contain the active ingredient in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents such as a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Compositions for injection are typically aqueous, and comprise a buffer, e.g. citrate buffer. No other ingredients may be required. The pH of such a composition may be, for example from 4 to 7, e.g. about 5.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are known.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate.

An intravenous unit dose of amisulpride suitable for use in the invention is preferably a single injection containing amisulpride. In a preferred embodiment, this could be in the form of a vial of the active agent(s) along with a syringe and needle or a prefilled syringe/needle combination.

In some embodiments, the amisulpride is in a non-IV injectable formulation. It may be in the form of a solid or liquid formulation, and may be formulated for oral administration. The solid formulations may be in the form of a tablet or capsule, a melt tablet, or in the form of a dispersible powder or granules (that may need to be added to water). Liquid formulations may be in the form of an aqueous or oily suspension or in the form of a syrup, and they may be packaged in a vial.

Amisulpride may be in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols.

For topical delivery, transdermal and transmucosal patches, creams, ointments, jellies, solutions or suspensions may be employed. For sub-lingual delivery, fast dissolving tablet formulations may be used, as well as a number of the presentations described above. For oral administration, which is preferred amisulpride may be administered as tablets, capsules or liquids.

In a preferred embodiment, an oral unit dose of amisulpride is in the form of one of more tablets, or one or more capsules. The unit doses of amisulpride may be provided in a blister pack.

Amisulpride formulations may contain any number of pharmaceutically acceptable excipients, such as sweeteners and preservatives.

Formulations of amisulpride suitable for use in the invention are described in WO2011/110854.

Where a use or a method of the invention provides for the administration of more than one drug, they can be administered simultaneous, sequentially or separately. It is not necessary that they are packed together (but this is one embodiment of the invention). It is also not necessary that they are administered at the same time. As used herein, “separate” administration means that the drugs are administered as part of the same overall dosage regimen (which could comprise a number of days), but preferably on the same day. As used herein “simultaneously” means that the drugs are to be taken together or formulated as a single composition. As used herein, “sequentially” means that the drugs are administered at about the same time, and preferably within about 1 hour of each other.

As used herein, “therapy” means treatment or prevention. Preferably, the amisulpride for use in the invention is used in the prevention of postoperative emesis.

In some embodiments, amisulpride according to the present invention is useful in patients undergoing a surgical procedure where postoperative emesis would be potentially dangerous to the patient. For example, an incidence of emesis in these patients could cause hazardous medical complications that are potentially fatal to the patient such as emesis causing sutures to rupture and thereby resulting in a patient bleeding out or allowing a serious infection to take hold.

Further examples of these dangerous/hazardous medical complications caused by postoperative emesis are aspiration into the lungs, suture dehiscence, oesophageal rupture, subcutaneous emphysema, bilateral pneumothoraxes venous hypertension, increased intracranial pressure, or hematomas such as those beneath surgical flaps, vascular anastomoses, and aneurysm clips.

The skilled person will be aware of the surgical procedures in which postoperative emesis would be problematic (or would lead to the complications described above). Examples of these surgical procedures are surgery of the mouth cavity (such as wired jaw surgery or dental surgery), surgery of the ear, nose or throat (ENT) (such as tonsillectomy or thyroidectomy), surgery of the head or face (such as craniotomy, hemorrhagic stroke surgery, ischemic stroke surgery, rhinoplasty, a cosmetic procedure of the face or eye surgery), surgery of the gastrointestinal (GI) tract (such as paraesophageal surgery, anti-reflux surgery, bariatric surgery, gastrectomy, gastric bypass surger or gastric sleeve surgery), lung surgery (such as a surgical lung biopsy, lobectomy or a wedge resection), abdominal surgery (such as a surgical hernia repair, a total abdominal hysterectomy, abdominoplasty, laparotomy, any surgery involving a large abdominal incision, or an open abdominal aortic aneurysm repair) or bowel surgery.

Preferably, a surgical procedure according to the invention involves the administration of a general anaesthesia e.g. general inhalation anaesthesia. The procedure may be an elective surgery (open or laparoscopic technique) under general anaesthesia. It is preferably scheduled to last at least one hour from induction of anaesthesia to extubation. Prior to extubation, the wound will be closed.

As used herein “undergoing a surgical procedure” means the time period from about 2 hours preceding the surgical procedure until an episode of emesis in the period of about 24 hours following the surgical procedure (at which stage the therapy ceases to be prevention and is classed as treatment).

It is preferred that the amisulpride is used in the prevention of postoperative emesis, i.e. it is administered as described above, but before an incidence of emesis occurs. It is preferably administered as a single prophylactic dose.

In a preferred embodiment, the amisulpride is administered up to 4 hours before the surgical procedure. It is preferably administered no later than at the time of wound closure/end of surgery, more preferably at the time of anaesthesia (and more preferably, at the time of induction of the anaesthesia).

Preferably, the amisulpride is administered by IV infusion (push), preferably over a time period of from about 20 seconds up to 1 or 2 minutes. In some embodiments, this period may be up to 10 minutes, for example, if the patient has pain on injection or where a higher dose (e.g. 20 mg) is being administered. In a preferred embodiment, the amisulpride is administered over about 1 to 2 minutes, or 1 or 2 minutes. The amisulpride is preferably administered in a single dose.

Preferably, the patient has at least 3 risk factors for post-operative emesis, wherein the risk factors are selected from a past history of postoperative nausea and vomiting and/or motion sickness; habitual non-smoking status; being a female; and expected use of post-operative opioid analgesia. More preferably, the patient has all four risk factors. These risk factors may define a patient group for which amisulpride is particularly useful in the therapy of post-operative emesis.

In a particularly preferred embodiment of the invention, amisulpride at a dose of 5 mg is useful in the prevention of postoperative emesis in a patient, preferably wherein the patient is undergoing a surgical procedure where postoperative emesis would be potentially dangerous to the patient, and wherein the patient has at least three risk factors for post-operative emesis, wherein the risk factors are selected from a past history of postoperative nausea and vomiting and/or motion sickness; habitual non-smoking status; being a female; or expected use of post-operative opioid analgesia.

The following study illustrates the invention.

### Study 1

### Protocol

A randomised, double-blind, placebo-controlled Phase Ill study of amisulpride as combination prophylaxis against post-operative nausea and vomiting in high-risk patients was conducted. The primary aim of the study was to assess the efficacy of amisulpride at 5 mg in the prevention of post-operative nausea and vomiting (PONV) in adult, surgical patients at high risk of PONV. The amisulpride was administered in combination with a standard anti-emetic.

The subjects of the study were 1204 randomised adult patients (18 years) at high risk of PONV who were undergoing elective ambulatory (day-case) or in-patient surgery under general inhalational anaesthesia for an expected duration of at least one hour from induction of anaesthesia to extubation. Of the 1204 randomised patients, 1147 were dosed and eligible.

High risk of PONV was defined as having at least three of the following risk factors:
Past history of PONV and/or motion sickness
Habitual non-smoking status
Female
Expected use of post-operative opioid analgesia

5 mg amisulpride was administered as a single, slow, intravenous (IV) push over one minute at the time of induction of anaesthesia; given in combination with a standard anti-emetic. Examples of standard anti-emetics used were ondansetron, granisetron and dexamethasone.

The two arms of the study were as follows:
Arm 1: amisulpride IV at 5 mg in combination with a standard anti-emetic which is not a dopamine D2-antagonist (e.g. ondansetron IV at 4 mg, granisetron IV at 1 mg, or dexamethasone IV at 4 mg);
Arm 2 (control): amisulpride IV placebo in combination with a standard anti-emetic, as defined above
The primary efficacy variable was the absence or presence of PONV during the 24-hour post-operative period, where PONV was defined as the occurrence of one or more emetic episodes (vomiting and/or retching) or the receipt of one or more doses of rescue anti-emetic medication. Absence of PONV by this definition was termed “Complete Response” (CR). A number of secondary variables were evaluated including the occurrence of emesis (vomiting and/or retching).

### Primary Efficacy Analysis

A comparison of the incidence of CR in the 0-24-hour period after surgery between the amisulpride group and the placebo group using Pearson's x2 test with Yates's continuity correction at a one-sided significance level of 2.5%. The primary efficacy analysis population was the modified intent-to-treat (mITT) population.

### Secondary Efficacy Analyses

Secondary efficacy variables assessed by incidence (e.g., emesis) were compared between the groups using Pearson's x2 test.

### Results (Extract)

A summary of the data upon which the present invention is based is a follows:

TABLE 1
Incidence of PONV over a 24 hour
period - RRR (relative risk reduction)

| | Total | Number of | Number of | % |
|---|---|---|---|---|
| | number of | patients | patients | patients |
| | patients | with CR* | with PONV | with PONV |
| Placebo plus | 575 | 268 | 307 | 53.39 |
| a standard | | | | |
| anti-emetic** | | | | |
| Amisulpride 5 | 572 | 330 | 242 | 42.31 |
| mg plus a | | | | |
| standard | | | | |
| anti-emetic** | | | | |
| Difference in | | | | 11.08 |
| PONV rate | | | | |
| Calculated | | | | 20.76 |
| RRR | | | | |

*complete response
**examples of standard anti-emetics listed above

TABLE 2
Incidence of post-operative emesis
(vomiting and/or retching) - RRR

| | | Number of | % |
|---|---|---|---|
| | | patients | patients |
| | Total | with post- | with post- |
| | number of | operative | operative |
| | patients | emesis | emesis |
| Placebo plus a | 575 | 115 | 20.00 |
| standard | | | |
| anti-emetic** | | | |
| Amisulpride | 572 | 79 | 13.81 |
| 5 mg plus a | | | |
| standard | | | |
| anti-emetic** | | | |
| Difference in | | | 6.19 |
| post-operative | | | |
| emesis rate | | | |
| Calculated | | | 30.94 |
| RRR | | | |

### CONCLUSION

Amisulpride plus standard antiemetic improved the CR rate versus placebo plus standard antiemetic by 11.08% in the mITT (modified intention to treat) population which was statistically significant (p<0.001) and equated to a relative risk reduction (RRR) in the rate of PONV of 20.8%.

The occurrence of emesis (vomiting and/or retching) was statistically significantly lower (p=0.003) with the amisulpride group compared with placebo with a surprisingly high RRR of 31% compared to overall PONV RRR.

1.-24. (canceled)

25. A method for treating or preventing postoperative emesis in a patient, wherein the patient is undergoing a surgical procedure, comprising administering to the patient and effective amount of amisulpride, wherein:
the amisulpride is administered in combination with another anti-emetic, either separately, sequentially or simultaneously,
the dose of amisulpride is 5 mg, and
the patient has at least 3 risk factors for postoperative emesis, wherein the risk factors are selected from a past history of postoperative nausea and vomiting and/or motion sickness; habitual non-smoking status; being a female; and expected use of post-operative opioid analgesia.

26. The method according to claim 25, wherein the patient is undergoing a surgical procedure where postoperative emesis would be potentially dangerous to the patient.

27. The method according to claim 25, wherein the post-operative emesis would place the patient at risk of aspiration into the lungs, suture dehiscence, oesophageal rupture, subcutaneous emphysema, bilateral pneumothoraxes venous hypertension, increased intracranial pressure, or hematomas.

28. The method according to claim 27, wherein the hematomas are those beneath surgical flaps, vascular anastomoses, and aneurysm clips.

29. The method according to claim 25, wherein the surgical procedure is selected from surgery of the mouth cavity, surgery of the ear, nose or throat (ENT), surgery of the head or face, surgery of the gastrointestinal (GI) tract, lung surgery, abdominal surgery and bowel surgery.

30. The method according to claim 29, wherein the surgery of the mouth cavity is wired jaw surgery or dental surgery.

31. The method according to claim 29, wherein the ENT surgery is a tonsillectomy or thyroidectomy.

32. The method according to claim 29, wherein the surgery of the head or face is a craniotomy, hemorrhagic stroke surgery, ischemic stroke surgery, rhinoplasty, a cosmetic procedure of the face or eye surgery.

33. The method according to claim 29, wherein the GI tract surgery is paraesophageal surgery, anti-reflux surgery, bariatric surgery, gastrectomy, gastric bypass surgery or gastric sleeve surgery.

34. The method according to claim 29, wherein the lung surgery is a surgical lung biopsy, lobectomy or wedge resection.

35. The method according to claim 29, wherein the abdominal surgery is a surgical hernia repair, a total abdominal hysterectomy, abdominoplasty, laparotomy, any surgery involving a large abdominal incision, or an open abdominal aortic aneurysm repair.

36. The method according to claim 25, wherein the other anti-emetic is a 5HT₃ antagonist, an NK₁ antagonist or a steroid.

37. The method according to claim 25, wherein the other anti-emetic is dexamethasone, ondansetron, granisetron, palonosetron, aprepitant, netupitant or rolapitant.

38. The method according to claim 25, wherein the amisulpride is substantially in the form of a racemate.

39. The method according to claim 25, wherein the amisulpride is in the form of (S−)-amisulpride, which is substantially free of the (R+)-enantiomer.

40. The method according to claim 25, wherein the amisulpride is administered via the intravenous route.

41. The method according to claim 40, wherein the amisulpride is administered by infusion over about 1 to 2 minutes.

42. The method according to claim 25, wherein the amisulpride is administered in a single dose.

43. The method according to claim 25, wherein the amisulpride is administered at the time of induction of anaesthesia.
